(19) United States
(12) Patent Application Publication
(10) Pub. No.: US 2023/0000905 A1
(43) Pub. Date: Jan. 5, 2023

(54) IODINE AND POLYOL COMPOSITION, METHOD, AND USE

(71) Applicant: Janice Goodman, Toronto (CA)

(72) Inventor: Janice Goodman, Toronto (CA)

(21) Appl. No.: 17/779,230

(22) PCT Filed: Oct. 12, 2021
(86) PCT No.: PCT/CA2021/051430

§ 371 Date: May 24, 2022

Related U.S. Application Data
(60) Provisional application No. 63/090,861, filed on Oct. 13, 2020.

(51) Int. Cl. A61K 33/18; A61K 31/047; A61K 47/20; A61K 9/00

(52) U.S. Cl. CPC A61K 33/18; A61K 31/047; A61K 47/20; A61K 9/0073; A61K 9/0056; A61K 9/0043; A61K 9/0048

(57) ABSTRACT

A composition having molecular iodine in combination with a polyol can be used for control and treatment of microbes and infections on biological and non-biological surfaces. Compositions have a combination of molecular iodine (I2) and an acceptable source of iodate (IO3), and a buffering acid (inorganic or organic), in combination with a polyol in an amount of 3-50% by weight. The present compositions can also have prebiotic and probiotic properties on biological surfaces and membranes for the control and treatment of infection as well as the support of growth of commensal microbes and the control of pathogenic microbes.

### CROSS-REFERENCE TO RELATED APPLICATIONS

This application claims priority to United States provisional patent application U.S. 63/090,861 filed 13 Oct. 2020, which is hereby incorporated by reference herein in its entirety.

### FIELD OF THE INVENTION

The present invention pertains to a composition for control and treatment of pathogens. The present invention also pertains to a composition that has prebiotic and probiotic properties for the control and treatment of infection.

### BACKGROUND

Establishing and maintaining a healthy oral and membrane microbiome is important in assuring human homeostasis and health. Where diet and the microbiome helped to enable the development and evolution of humankind, dysbiosis has been the cause of many human diseases. Diet, use of synthetic antimicrobials, and exercise shape our microbiome and define us, however when the oral or nasal flora balance is deleteriously affected there can be a systemic price that is paid in shifting or disrupting the microbiome. (Eugene Rosenberg et al., Microbes Drive Evolution of Animals and Plants: the Hologenome Concept, mBio. 2016 Mar 31;7(2)). It has also been found that the microbiome of professional athletes differs from that of more sedentary subjects in composition and particularly at the functional metabolic level (S. F. Clarke et al., Exercise and associated dietary extremes impact on gut microbial diversity Gut 2014; 63:1913-1920). It is well established that certain periodontal bacteria are connected with human diseases such as dementia (P. gingivitis), cardiovascular disease, diabetes, and increased death rates. The care and treatment of the human body should thus be guided by the principal of returning to a healthy microbiome.

In the majority of plants, the main glucide transported is saccharose, but in certain plants, polyols are also transported. (F. W. Marlowe and J. C. Berbesque, (2009), Tubers as fallback foods and their impact on Hadza hunter-gatherers. Am. J. Phys. Anthropol., 140: 751-758.) There are significant differences between European children and rural African children in gut microbiota, with a high fiber African diet resulting in a population with an abundance of the bacteria from the genus Prevotella. (Carlotta De Filippo et al., Proc Natl Acad Sci USA. 2010 Aug 17; 107(33): pp. 14691-14696.) Polyols have been shown to act as prebiotics to these commensal bacteria and at the same time can hinder some of the pathogenic oral bacteria and result in growth inhibition for cariogenic bacteria. (Siin Koljalg et al., Scientific Reports volume 10, Article number: 6297, 2020). In particular, it has been found that oral treatment with a polyol results in a high growth inhibition effect on cariogenic bacteria.

At present time, current caries prevention therapy largely consists of fluoride therapy, which has been a ubiquitous halogen in dental products for several generations and is generally known to act to affect the surface hardness of teeth. However, it has been shown that fluoride can affect children's neurodevelopment and cause neurotoxicity at high levels. (A. Choi et al., Environ. Health Perspect. 2012 October; 120(10): 1362-1368). In addition, Jiang et al. showed that developmental neurotoxicity of fluoride may be closely associated with glucose utilization and glial acidic protein and increased levels of neurotrophic factor in the rat brains leading to neurodegenerative changes. (C. Jiang et al., Neuromolecular Med. 2014 Mar;16(1):94-105). Another study found that that mothers with high fluoride exposure in pregnancy had babies with associated lower IQ scores in 3 and 4 year olds (R. Green et al., JAMA Pediatr. 2019; 173(10):940-948). Many other concerns about the deleterious effects of fluoride on human health abound and are brought to our attention frequently. While fluoride has done some good in reducing caries largely by affecting the tooth, it does not address the root causes of the caries which is the modern diet, sedentary lifestyle, increase in non-obligate mouth breathing, and disruption of the oral microbiome.

The tiny radical nitric oxide (NO) participates in a vast number of physiological functions including vasodilation, nerve transmission, pain response, host defence and cellular energetics. Classically produced by a family of specific enzymes called NO synthases (NOSs), NO signals via reactions with other radicals or transition metals. An alternative pathway for the generation of NO is the nitrate-nitrite-NO pathway in which the inorganic anions nitrate (NO₃⁻) and nitrite (NO₂)⁻ are reduced to NO and other reactive nitrogen intermediates. Nitrate and nitrite are oxidation products from NOS-dependent NO generation but are also constituents in our diet, found mainly in leafy green vegetables. Various nitrate reducing bacteria are found in the mouth, including but not limited to Neisseria flavescens, Haemophilus parainfluenzae, Neisseria mucosa, Prevotella melaninogenica, Granulicatella adiacens, Veillonella dispar, Veillonella atypica, Veillonella parvula, Neisseria sicca, Prevotella salivae, Actinomyces odontolyticus, Actinomyces viscosus, Actinomyses oris, and Neisseria subflava. (Charlene E. Goh et al., Journal of the American Heart Association. 2019, Vol. 8, No. 23) Irrespective of origin, active uptake of circulating nitrate in the salivary glands, excretion in saliva, and subsequent reduction to nitrite by oral commensal bacteria are important steps for NO generation.

The central role of the oral cavity in regulating NO generation from nitrate presents a new and intriguing aspect of the human microbiome in health and disease. Recent studies have shown that nitrite and nitrate play an important role in cardiovascular and gastrointestinal homeostasis through conversion back into NO via a physiological system involving enterosalivary recirculation, bacterial nitrate reductases, and enzyme catalyzed or acidic reduction of nitrite to NO. (J. Jones et al., Pediatric Research, 2015 Jan; 77(0): pp. 173-181.) Nitrite synthesis by commensal oral bacteria is also a key mechanism to induce the vascular response to exercise over the first period of recovery thereby promoting lower blood pressure and greater muscle oxygenation. (C. Cutler et al.; Free Radical Biology and Medicine, Vol 141, November 2019, pp. 252-259) Oral bacteria in a healthy microbiome normally reduce enterosalivary nitrites, however when their amounts are reduced by routine mouthwash use there can be an increased risk for pre-diabetes and diabetes. (K. J. Joshipura et al., Nitric Oxide, Vol 71, December 2017, pp. 14-20) Broad spectrum antiseptic mouthwashes have also been found to reduce oral nitrite production by 90% and plasma nitrite levels by 25% compared to controls. (V. Kapil et al., Free Radical Biology and Medicine, Vol. 55, February 2013, pp.93-100)

With the use of probiotics it is possible to supplement humans with nitrates to encourage the endogenous NO synthesis cascade. This can result in a reduction in blood pressure, protection against cardiac ischemia reperfusion damage, restoration of nitric oxide homeostasis, and associated cardiovascular protection, increased vascular regeneration after chronic ischemia, and a reversal of vascular dysfunction in the elderly. While it may be a worthwhile procedure to bring back a healthy microbiome after disruption, it would be beneficial to sustain or increase the commensal bacteria in the first place while reducing the prevalence of harmful bacteria.

The monosaccharide xylitol has been used in nasal sprays to treat congestion, irritation, inflammation, and upper respiratory infections such a otitis media, sinusitis. In one example, United States patent U.S. Pat. No. 6,258,372 to Jones describes a nasal composition comprising by weight 100 parts of water, between 65 parts to 1 part of xylitol/xylose, and between 0.95 and 0.45 parts of sodium chloride.

Iodine has been in continuous use by the human race for 5,600 years when seaweed sponges were utilized to shrink goiters. Povidone iodine (PVP-I) released in 1955 is a brown, aqueous solution containing several different species of iodine (including iodide, iodate, triiodide, hypo-iodous acid and molecular iodine) bound to a large organic molecule, polyvinylpyrrolidone (povidone). Povidone, being highly soluble in water, carries the different iodine species, which have varying degrees of solubility, into solution along with it. 10% PVP-I (common brand:Betadine®) is used in hospitals, medical and dental offices worldwide and contains over 30,000 ppm of total iodine but only 1-3 ppm of molecular iodine which is the biocidal active species. All of the other species of iodine present in PVP-I contribute to staining, toxicity and irritation, but are not biocidal.

Molecular iodine (I₂) can be used as a topical antibiotic and is safe for human use, and stable in solution in a clear, almost tasteless, and biocidal product. In one example, United States patent U.S. Pat. No. 10,092,006 to Kolsky et al. describes an aqueous composition comprising a combination of molecular iodine (I₂), a source of iodate (IO₃), and an acid, that can be used as a disinfecting solution, germicide and/or biocide on various surfaces including living and inanimate surfaces. By maintaining iodate at a greater concentration than iodide and buffering it to a pH between 2.0-6.5, the molecular iodine is stabilized in solution for an extended period of up to 5 years. I₂ is also an essential element, natural, non-staining, organic, safe for chronic use, and is priced with the most affordable of all the antimicrobials. Kolsky et al. demonstrated that molecular iodine at the tested concentrations has the ability to inactivate rhinovirus, a resilient, non-enveloped virus largely responsible for upper respiratory infections in 30 seconds.

There remains a need for a composition that provides antibiotic activity for deleterious bacteria as well as probiotic activity to encourage commensal bacteria. There also remains a need for a non-toxic composition for surface treatment and control of infection caused by microbes, viruses, fungi, and other pathogens.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a composition for treatment and control of infection caused by microbes, viruses, fungi, and other pathogens. Another object of the present invention is to provide a topical composition that has prebiotic and probiotic properties for the control and treatment of infection. The aqueous composition comprises molecular iodine (I₂), a source of iodide (1), a source of iodate (IO₃), and a polyol, which can be used as a topical, internally, or surface treatment, preventative, and therapeutic.

In an aspect there is provided an aqueous composition comprising: a polyol; uncomplexed molecular iodine (I₂); a source of iodide (I⁻) in an effective amount; a source of iodate (IO₃) in an effective amount; and a buffering acid, wherein the buffering acid maintains the composition at a pH of between about 2.0 to about 6.5, and wherein iodide and iodate are present in the composition at a molar ratio of about 0.1 to about 25 and the composition provides a stable concentration of molecular iodine within the range of 0.5 ppm to 2500 ppm.

In an embodiment, the polyol is present in the composition in an amount of at least 3% by weight of the composition.

In another embodiment, the polyol is selected from mannitol, erythritol, sorbitol, dulcitol, galactitol, inositol, xylitol, lactitol, maltitol, ribitol, arabitol, and a combination thereof.

In another embodiment, the composition comprises more than one polyol.

In another embodiment, the polyol is xylitol, erythritol, or a combination of xylitol and erythritol.

In another embodiment, the xylitol and erythritol are in a 1:1 ratio in the composition.

In another embodiment, the composition further comprises flavour, color, glycerine, a whitening agent, a bleaching agent, a surfactant, breath freshener, or a combination thereof.

In another embodiment, the surfactant is selected from a nonionic surfactant, an anionic surfactant, a cationic surfactant, an amphoteric surfactant, a zwitterionic surfactant, and a combination thereof.

In another embodiment, the surfactant is an alkyl sulfate, alkylether sulfate, alkyl benzene sulfonate, alpha olefin sulfonate, N-alkyl sarcosinate, alkyl sulfosuccinate, alkyl phosphate, alkylether phosphate and alkyl carboxylic acid salt, or alkylether carboxylic acid salt.

In another embodiment, the composition is formulated as a spray, inhalant, liquid, gel, or lozenge.

In another embodiment, the composition is formulated for nasal application, oral application, inhalation, eye application, or aural application.

In another embodiment, the polyol is present in the composition in an amount of between 3-30% by weight.

In another embodiment, the source of iodide is selected from the group consisting of sodium iodide, potassium iodide, lithium iodide, calcium iodide, magnesium iodide, hydroiodic acid and mixtures thereof and the source of iodate is selected from the group consisting of sodium iodate, potassium iodate, lithium iodate, calcium iodate, magnesium iodate, hydroiodic acid, and mixtures thereof.

In another embodiment, the source of iodate is sodium iodate, potassium iodate, and a mixture thereof.

In another embodiment, the composition further comprises hydroiodic acid.

In another embodiment, the concentration of uncomplexed molecular iodine ranges from 1 ppm to 500 ppm in the composition.

In another aspect there is provided a method of topical disinfection comprising applying to a surface a composition comprising: a polyol; uncomplexed molecular iodine (I₂); a source of iodide (I⁻) in an effective amount; a source of iodate (IO₃) in an effective amount; and a buffering acid; wherein the buffering acid maintains the composition at a pH of between about 2.0 to about 6.5, and wherein iodide and iodate are present in the composition at a molar ratio of about 0.1 to about 25 and the composition provides a stable concentration of molecular iodine within the range of 0.5 ppm to 2500 ppm.

In an embodiment, the composition is applied in an oral cavity, nasal cavity, ear, eye, or on skin.

In another embodiment, the surface is a mucous membrane, human surface, animal surface, agriculture surface, biological membrane, dental surface, skin, medical device, commercial surface, laboratory surface, or industrial surface.

In another embodiment, the polyol is selected from mannitol, erythritol, sorbitol, dulcitol, galactitol, inositol, xylitol, lactitol, maltitol, ribitol, arabitol, and a combination thereof.

In another aspect there is provided a use of a composition comprising: applying to a surface a disinfecting amount of a composition comprising: a polyol; uncomplexed molecular iodine (I₂); a source of iodide (I⁻) in an effective amount; a source of iodate (IO₃) in an effective amount; and a buffering acid; wherein the buffering acid maintains the composition at a pH of between about 2.0 to about 6.5, and wherein iodide and iodate are present in the composition at a molar ratio of about 0.1 to about 25 and the composition provides a stable concentration of molecular iodine within the range of 0.5 ppm to 2500 ppm.

In an embodiment, the surface is floss, a wipe, a cotton swab, a Q-tip™, a brush, a toothbrush, a tongue cleaner, a toothpick, an interproximal cleaner, an applicator, a bandage, tape, or gauze.

In another embodiment, the use is agricultural, medical, dental, environmental, or veterinary.

In another embodiment, the polyol is selected from mannitol, erythritol, sorbitol, dulcitol, galactitol, inositol, xylitol, lactitol, maltitol, ribitol, arabitol, and a combination thereof.

### DETAILED DESCRIPTION OF THE INVENTION

Unless defined otherwise, all technical and scientific terms used herein have the same meaning as commonly understood by one of ordinary skill in the art to which this invention belongs.

The use of the word “a” or “an” when used herein in conjunction with the term “comprising” may mean “one,” but it is also consistent with the meaning of “one or more,” “at least one” and “one or more than one.” Thus, for example, reference to a “source of iodine” includes a single source as well as two or more different sources, reference to an “active” refers to a single as well as to two or different actives, reference to an “inert” includes a single excipient as well as two or more different inerts, and the like.

As used herein, the terms “comprising,” “having,” “including” and “containing,” and grammatical variations thereof, are inclusive or open-ended and do not exclude additional, unrecited elements and/or method steps. A composition, device, article, system, use, or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to. Where a range of values is provided, it is understood that each intervening value between the upper and lower limit of that range and any other stated or intervening value in that stated range is encompassed within the invention. Where the stated range includes one or both of the limits, ranges excluding either both of those included limits are also included in the invention.

The term “compound”, as used herein, unless otherwise indicated, refers to any specific chemical compound disclosed herein. Within its use in context, the term generally refers to a single compound as otherwise described herein. Compounds which are disclosed are those which are stable.

The term “effective” is used, in context, to describe an amount or concentration of a compound, composition or component, as otherwise described herein which is included or used to provide an intended effect or trait as otherwise described in context, such as disinfection, biocidal and/or antimicrobial activity, or other attribute, such as buffering effect, depending upon the final composition, or an effect or trait dependent upon the nature of the final product such as surfactancy, emulsification (emulsifiers), emolliency, wetability, skin adherence, storage stability, and/or solubility to a formulation or to produce a compound or composition according to the present invention. It is noted that when the term “effective” is used within the context of disinfection with molecular iodine according to the present invention, this term is used to describe an effective amount of the composition containing an effective amount of uncomplexed molecular iodine which is contacted with the surface to be disinfected for a period of time and at a temperature (often at room temperature, but in certain embodiments at elevated temperatures such as at 37 degrees to about 50 degrees celcius or more) sufficient to disinfect the membrane or surface.

The term “source of iodide” is used to refer to an appropriate compound or material which provides an effective concentration of iodide anion in solution which is used in compositions according to the present invention. The source of iodide used in compositions according to the present invention includes any appropriate source of iodide, such as iodide salts (and includes hydroiodic acid) which dissociate when placed in solution. Preferred sources of iodide for use in the present invention include but are not limited to Nal (sodium iodide), KI (potassium iodide), Lil (lithium iodide), Cal₂ (calcium iodide) and MgI₂ (magnesium iodide).

The term “source of iodate” is used to refer to an appropriate compound or material which provides a concentration of iodate anion in solution which is used in the present invention. The source of iodate used in compositions according to the present invention includes any appropriate source of iodate, such as iodate salts which dissociate when put in solution. Sources of iodate for use in the present invention include but are not limited to NaIO₃ (sodium iodate), KIO₃ (potassium iodate), LilO₃ (lithium iodate), CaIO₃ (calcium iodate), and MgIO₃ (magnesium iodate).

The term “disinfect” as used herein refers to significantly reducing and/or effectively eliminating microbes or pathogens from membranes or surfaces to which the presently described compositions are applied or solutions in which the compositions are added to a level of at least about 99%, preferably at least about 99.9%, preferably at least about 99.99%, preferably at least about 99.999%, or greater, preferably where the remaining population of pathogen is less than about 10⁻⁶ or even lower, including to a level such that the pathogens initially in solution or on the membrane treated are eliminated to a level beyond the capability of contemporary analysis (i.e., they are essentially eliminated from the surface treated with compositions according to the present invention).

The term “antimicrobial” as used herein refers to compositions that have activity against a broad range of microscopic organisms and biological molecules including but not limited to viruses, bacteria, fungi, spores, mycobacteria, parasites, prions, and other microbes. It is understood that microbes can be commensal or beneficial to humans and other animals, neutral (neither harmful nor beneficial), or deleterious (harmful to humans or other animals), also referred to as pathogenic.

The term “antiviral” is used herein to describe the activity of compositions according to the present invention which display general activity against viruses including animal, plant, fungal and bacterial viruses. Viruses which may be inhibited and/or eliminated pursuant to the methods according to the present invention using compositions disclosed herein include those which impact animals, especially mammals, and in particular humans, fish, and domestic animals. Viruses for which the presently described compositions can display antiviral activity include but are not limited to: papovaviruses, e.g. Polyoma virus and sv40; poxviruses, e.g. Vaccinia virus and variola (smallpox); adenoviruses, e.g., human adenovirus; herpesviruses, e.g. Human herpes simplex types I and II; parvoviruses, e.g. Adeno associated virus (aav); reoviruses, e.g., rotavirus and reovirus of humans; picornaviruses, e.g. Poliovirus; togaviruses, including the alpha viruses (group a), e.g. Sindbis virus and semliki forest virus (sfv) and the flaviviruses (group b), e.g. Dengue virus, yellow fever virus and the St. Louis encephalitis virus; retroviruses, e.g. Human immunodeficiency virus (I and II); rous sarcoma virus (rsv), and mouse leukemia viruses; rhabdoviruses, e.g. vesicular stomatitis virus (vsv) and rabies virus; paramyxoviruses, e.g. mumps virus, measles virus and sendai virus; arena viruses, e.g., lassa virus; bunyaviruses, e.g., bunyawere (encephalitis); coronaviruses, e.g. common cold (rhinovirus) and severe acute respiratory syndrome coronavirus 2; gastrointestinal distress viruses; orthomyxovirus; caliciviruses, e.g. norwak virus; hepatitis E virus; filoviruses, e.g., ebola virus and marburg virus; astroviruses; influenza virus; Herpes Simplex Virus (HSV1 and HSV-2); Coxsackie virus; Human immunodeficiency virus (I and II); Andes virus; Papilloma virus; Epstein-Barr virus (Human gammaherpesvirus); Variola (smallpox) and other pox viruses; West Nile virus. Animal viruses that may also be relevant targets of compositions according to the present invention include: Norovirus; Reovirus; Rotavirus; Aphthovirus; Parechovirus; Erbovirus; Kobuvirus; Teschovirus; Enterovirus; Rhinovirus; Hepatovirus; Hepatitis E virus; Rubella virus; Lymphocytic choriomeningitis virus; HIV-1; HIV-2; HTLV-I Herpes Simplex Virus 1 and 2; Cardiovirus; Norwalk virus; Influenzavirus A, B and C; Isavirus; Thogotovirus; Coxsackie Virus; Dengue virus; Yellow fever virus; Hepatitis A virus; Hepatitis B virus; Hepatitis C virus; Measles virus; Mumps virus; Respiratory syncytial virus; California encephalitis virus; Hantavirus; Rabies virus; Ebola virus;Marburg virus; Corona virus; Astrovirus; Borna disease virus; and Variola (smallpox virus). Plant viruses can also be relevant targets of compositions according to the present invention include but are not limited to the following: Partitiviruses, e.g., alphacryptoviruses and betacryptoviruses; Potyviruses, e.g., bymoviruses and ipomoviruses; Bromoviruses, e.g. cucumoviruses and bromoviruses; Comoviruses, e.g. fabiviruses, neopoviruses and comoviruses; Geminiviruses e.g., bigeminivirus, monogeminivirus and bybrigeminivirus; Rhabodoviruses, e.g., cytorhabdoviruses, nucleorhabdoviruses; Reoviruses, e.g., oryzaviruses and phytoreoviruses; Satellite viruses, e.g., satelliviruses; Tombusviruses, e.g., carmoviruses; Sequiviruses, e.g., sequiviruses and waikaviruses; among numerous others, including those listed hereinbelow. The present invention may be used to disinfect, eliminate and/or inhibit the growth of plant viruses, especially in certain agricultural applications, especially including food production. Plant Virus Genuses which can also be targets of the present compositions and methods include but are not limited to: Partitiviruses, e.g., alphacryptoviruses and betacryptoviruses; Potyviruses, e.g., bymoviruses and ipomoviruses; Bromoviruses, e.g. cucumoviruses and bromoviruses; Comoviruses, e.g. fabiviruses, neopoviruses and comoviruses; Geminiviruses e.g., bigeminivirus, monogeminivirus and bybrigeminivirus; Rhabodoviruses, e.g., cytorhabdoviruses, nucleorhabdoviruses; Reoviruses, e.g., oryzaviruses and phytoreoviruses; Satellite viruses, e.g., satelliviruses; Tombusviruses, e.g., carmoviruses; Sequiviruses, e.g., sequiviruses and waikaviruses. Other plant virus genuses which are targets of the present compositions and methods, include but are not limited to: Alfamoviruses, Bromoviridae; Alphacryptoviruses, Partitiviridae; Badnaviruses; Betacryptoviruses, Partitiviridae; Bigeminiviruses, Geminiviridae; Bromoviruses, Bromoviridae; Bymoviruses, Potyviridae; Capilloviruses; Carlaviruses; Carmoviruses, Tombusviridae; Caulimoviruses; Closteroviruses; Comoviruses, Comoviridae; Cucumoviruses, Bromoviridae; Cytorhabdoviruses, Rhabdoviridae; Dianthoviruses; Enamoviruses; Fabaviruses, Comoviridae; Fijiviruses, Reoviridae; Furoviruses; Hordeiviruses; Hybrigeminiviruses, Geminiviridae; ldaeoviruses; Ilarviruses, Bromoviridae; Ipomoviruses, Potyviridae; Luteoviruses; Machlomoviruses; Macluraviruses; Marafiviruses; Monogeminiviruses, Geminiviridae; Nanaviruses; Necroviruses; Nepoviruses, Comoviridae; Nucleorhabdoviruses, Rhabdoviridae; Oryzaviruses, Reoviridae; Ourmiaviruses; Phytoreoviruses, Reoviridae; Potexviruses; Potyviruses, Potyviridae; Rymoviruses, Potyviridae; Satellite RNAs; Satelliviruses; Sequiviruses, Sequiviridae; Sobemoviruses; Tenuiviruses; Tobamoviruses; Tobraviruses; Tombusviruses, Tombusviridae; Tospoviruses, Bunyaviridae; Trichoviruses; Tymoviruses; Umbraviruses; Unassigned potyviruses, Potyviridae; Unassigned rhabdoviruses, Rhabdoviridae; Varicosaviruses; Waikaviruses, Sequiviridae; and other ungrouped plant viruses.

The term “antibacterial” as used herein refers to components and/or compositions that can be used to eliminate or significantly affect the number, health, or pathogenicity of numerous types of bacteria, including gram negative and gram positive bacteria, and including drug and multidrug resistant bacteria, including methicillin resistant staphylococcus Aureus (MRSA). Bacteria which are targets of the antimicrobial activity of compositions according to the present invention include but are not limited to Gram positive and gram negative bacteria including cocci and bacilli. Gram Positive bacteria that can be a target of the present composition include but are not limited to: Staph aureus; S. epidermidis; S. saphrophyticus; S. haemolyticus; S. hominis; S. capitis S. schleiferi; S. warneri; S. lugdenenis; Strep pyrogenes (gr. A); S. agalactiae (gr. B); E. faecalis; E. faecium; Enterococci; S. pneumoniae; S. mutans group; S. salivarus group; S. sanguis group; S. mitis group; S. angiosus group Abiotrophica defective; A. adiacens; S. milleri; S. bovis; N. gonorrhea; N. meningitides; Moraxella catarrhalis; C. diptheriae; C. jeikenium; C. urealyticum; Lactobacillus sp.; Bacillus anthracis; B. cereus; Listeria monocytogenes; Erisipelothrix rhusiopathiae; and Arcanobacterium bemolyticum. Gram Negative bacteria that can be a target of the present composition include but are not limited to: Escherichia coli; Klebsiella pneumoniae; Proteus spp.; Morganella; Providencia; Salmonella enterica; Shigella boydii (serogroup C); S. dysenteriae (serogroup A); S. flexneri; S. sonnei (serogroup D); C. freundii; C. koseri; Enterobacter cloacae; E. aerogenes; S. marcecescens; Vibrio cholera; V. parahaemolyticus; V. vulificans; Aeromonas hydrophila; Plesiomonas shigelloides; Acinetobacter baumannii; A. lowfii; Stenotrophomonas maltophilia; Pseudomonas sp; Pseudomonas aeruginosa; P. fluroescens; P. putida; Burkholderia cepacia; Alkaligenes; Haemophilus; H. influenzae; H. parainfluenzae; H. duceyi; HACEK group; Haemophilus aphrophilus; Actinobacter actinomysetemcomitans; Cariobacter hominis; Eikenella corrodens; Kingella kingii; Bordatella pertussis; Pasteurella multocida; Brucella sp.; Campylobacter; C. jejuni C. coli; C. fetus; Capnocytophaga; Francisella tularensis; Helicobacter pylon; Legionella pneumophila; Mycoplasma pneumoniae; M. hominis; Ureaplasma urealyticum; Bacteroides fragilis group; B. fragilis; B. distansonis; B. thetaiotaomicron; B. uniformis; Proteus vulgaris; B. ovatus; B. uniformis; Bacteroides sp; B. ureolyticus Bilophila wadsworthia Porphyromonas species; Prevotella; Fusobacterium; Clostridium sp; C. perfringens; C. botulinum; C. tetani; C. septicum; C. difficile; Actinomyces israeli; Propionibacterium acnes; Eubacterium; Lactobacillus sp.; Bifidobacterium; Veillonella; Peptostreptococcus; and Peptococcus.

The term “spores” is used herein to describe a unit of asexual reproduction and/or resistance of many plants, algae, fungi, bacteria and protozoa that are adapted survival and dispersal of these organisms in unfavorable conditions. Spores are usually unicellular and under favorable conditions can develop into a new organism. Spores may be characterized more specifically as sporangiospores from fungi, zygospores from fungi, ascospores from ascomycetes, basidiospores from basidiomycetes, aeciospores, teliospores and uredeiospores from fungi such as rusts or smuts, oospores from oomycetes, carpospores and tetraspores from red algae. The term spores also includes meiospores, microspores, megaspores, mitospores, zoospores, aplanospores, autospores, ballistospores and statismospores, among others.

The term “mold” is used herein to describe a fungus that grows in the form of multicellular filaments called hyphae. Molds are a large and taxonomically diverse number of fungal species where the growth of hyphae results in discoloration and a fuzzy appearance, especially on food. Molds are considered to be microbes and can generally found in the divisions of Zygomycota and Ascomycota. Molds often cause biodegradation of natural materials, which can be unwanted when it becomes food spoilage and/or damage to property. Molds also cause disease in animals and humans often resulting from allergic sensitivity to mold spores, from growth of pathogenic molds within the body, or from the effects of ingested or inhaled toxic compounds (mycotoxins) produced by molds. There are thousands of known species of molds, which have diverse life-styles including aprotrophs, mesophiles, psychrophiles and thermophiles and some are opportunistic pathogens of humans. Molds require moisture for growth and some live in aquatic environments. Like all fungi, molds derive energy from the organic matter on which they live, utilizing heterotrophy. Typically, molds secrete hydrolytic enzymes, which degrade complex biopolymers such as starch, cellulose and lignin into simpler substances which can be absorbed. In this way molds play a major role in causing decomposition of organic material, enabling the recycling of nutrients. Molds often grow on stored food for animals and humans, making the food unpalatable or toxic and are thus a major source of food losses and illness. Many prior art strategies (salting, pickling, jams, bottling, freezing, drying) are used to prevent or slow mold growth as well as growth of other microbes. Molds reproduce by producing large numbers of small spores, which can be inhibited and/or eliminated by the compositions according to the present invention. Common molds include Acremonium, Alternaria, Aspergillus, Cladosporium, Fusarium, Mucor, Penicillium, Rhizopus, Trichoderma and Stachybotrys, among others.

The term “membrane” as used herein refers to any biological surface to which compositions according to the present invention can be applied and on which may be used for its disinfectant, antimicrobial and/or biocidal activity. Membranes on which the present compositions can be used include but are not limited to keratinous surfaces such as the skin, hair or nails (ungual), mucosal surfaces (including internal surfaces of an animal or human, such as the throat, mouth (including teeth and/or gums) or nasal passages or other mucosal surfaces in the body including the ears, vagina or internal surfaces, or a wound of a patient or subject. Membranes can be, for example, in a human, animal, or plant. In certain applications, the compositions may be used internally on internal membranes in a patient or subject, for example, pursuant to medical procedures. Membranes also include hands and/or other body surfaces of a subject or patient, including skin surfaces, optionally in which incisions are to be made pursuant to surgical procedures. Other applications include direct application to mucosal and subgingival surfaces, ear drops and toothpaste. In addition, it is anticipated that the compositions described herein shall be directly applied to mucosal surfaces, e.g. douche, oral irrigation, lavage, oral ingestion, throat spray or gargle, nasal/sinus spray, paste, gel, or mouthwash.

The term “surface” as used herein refers to non-biological barriers or inanimate objects which can be treated with the present compositions. In particular, the present compositions can be used to disinfect inanimate objects such as, for example, medical devices, medical equipment, food preparation surfaces, food storage containers, plumbing lines, air supply conduits, and other surfaces. The term “surface” also refers to medical devices which can be coated with the present compositions to prevent, inhibit, or control surface growth of pathogens and/or can be used to apply the composition to surfaces or membranes.

As used herein, the term “polyol” is term of art that refers to an organic compound containing one hydroxyl group (—OH) attached to each carbon atom. Preferably the polyol is a sugar alcohol which has 4-6 carbons. Non-limiting examples of polyols which can be used with the present composition include mannitol, erythritol, sorbitol, dulcitol (galactitol), inositol, xylitol, lactitol, ribitol, and arabitol. Other polyols can be xylose, ribose, and other straight-chained or cyclic polyols with fewer than one hydroxyl per carbon atom or with one or more additional functional groups. The polyol can be naturally-occurring or synthetic. Sugar alcohols, compared to dietary sugars, are generally non-cariogenic (sugar-free tooth-friendly), low-glycaemic (potentially helpful in diabetes and cardiovascular disease), low-energy, low-insulinaemic (potentially helpful in obesity), and low-digestible.

As used herein, the term “molecular iodine” (I₂) or “uncomplexed molecular iodine” refers to diatomic iodine, which is a molecule comprised of two iodine atoms and is represented by the chemical symbol I₂. Some of the prior art uses the term “elemental iodine” to describe the same chemical entity.

The term “uncomplexed molecular iodine” or “free molecular iodine” refers to molecular iodine which is in free form in solution. In compositions according to the present invention at least about 50% (at least about 55%, at least about 60%, at least about 65%, at least about 70%, at least about 75%, at least about 80%, at least about 85%, 90%, 95% or more) of the total iodine species present are in uncomplexed molecular form and contribute to the chemical activity of molecular iodine as compared to a pure composition of molecular iodine in a 0.1 N HCl solution as measured by the potentiometric method of Gottardi (W. Gottardi, Arch Pharm, 1999. 332 (5): p. 151-157; W. Gottardi, Anal Chem 1983, 314, p.582-5).

The term “iodate” or “iodate anion” refers to the anion that is the conjugate base of iodic acid wherein an atom of iodine is bonded to three oxygen atoms. Iodate is represented by the chemical symbol IO₃⁻.

The term “chemical activity” refers to a measure of the effective concentration of molecular iodine when in the presence of other chemical species. The difference between the chemical activity of molecular iodine and the concentration of molecular iodine in iodophors is largely a measure of complexation of molecular iodine in iodophors.

The term “buffer” or “buffering acid” is used to describe any compatible inorganic or organic acid which is capable of maintaining a pH of compositions according to the present invention within the range of about 1.0 to about 6.5, about 1.5 to about 6.5, about 2.0 to about 5.5, about 2.0 to about 5.0, about 1.0 to about 3.5-4.0 with a preferred range of about 2.0 to about 4.0. The preferred pH of the compositions contemplated in the current invention will vary based upon the use regimen for an application. Buffering acids for use in the present invention include but are not limited to mono or polyacids, phosphate acids (including polyphosphate acids) such as phosphoric acid and its related salts sodium dihydrogen phosphate and sodium monohydrogen phosphate, polyphosphoric acid (Hₙ₊₂PₙO₃ₙ₊₁), organic acids having from two to 20 or more carbon atoms, including carboxylic acids according to the chemical structure R—CO₂H, where R is an optionally substituted, optionally branched, C₁-C₂₀ alkyl, alkenyl, alkynyl, aryl, or other carbon containing group optionally having more than one double bond or carboxylic acid moiety, and various sulphonic acids according to the chemical structure R₁—SO₃H, where R₁ is an optionally substituted, optionally branched, C₁-C₂₀ alkyl, alkenyl, alkynyl, aryl, or other carbon containing group optionally having more than one double bond. Organic acids which may be preferred for use in the present invention include, for example, citric, fumaric, glycolic, lactic, malic, tartaric, acetic, formic, oxalic acid, propanoic, propandioic, butanoic, butanedioic, pentanoic (valeric), pentandioic, hexanoic, hexandioic and benzoic, among others. Other acids which also may be used include, for example, acids of bisulfate (sodium, potassium bisulfate), sulfamic acid, and ethylenediaminetetraacetic acid, among others. The use of citric acid, phosphoric acid or other polyacids may be preferred because of the ability of these acids to accommodate a number of hydrogen ions in a single chemical entity, which may assist in maintaining the pH of compositions according to the present invention within a relatively narrow buffered range, thus maintaining activity and stability of the present compositions. A particularly preferred acid, citric acid, refers to the free acid or monobasic (e.g. sodium salt) form of 2-hydroxypropane-1,2,3-tricarboxylic acid (CAS Registry Number: 77-92-9). Compositions anticipated in the present invention can incorporate the di- and tribasic forms of citric acid provided there is an effective amount of the free acid in order to insure that the pH of the compositions lie within a range from about 1.5 to about 6.5, often about 2.0 to 5.5, most often about 2.0 to about 5.0.

The terms “stable” and “stabilized” as used herein refer to a composition according to the present invention that can be placed into normal distribution channels, and used as a commercial product with a minimum activated use-life of at least 1 week, or 1 month and preferably at least 4 to 6 months and most preferably with a activated use-life of 2-5 years with no substantial loss of thiosulfate titratable iodine-thereby maintaining activity as a disinfectant/germicide/biocide/antimicrobial composition.

The term “thickening agent”, “gelling agent” or “thickener” is used to describe a component which may be included in compositions according to the present invention to increase the viscosity of the composition to make the composition more readily adhere to a surface or membrane, such as teeth, nasal passages, throat, a ceiling, a vertical surface, or a surface which is present on an incline. Gelling agents for use in the present invention include standard gelling agents which are stable to acid solutions and which limit degradation due to oxidation. It is understood that gelling agents or thickeners for use in the present compositions that are used with biological membranes or in applications in which humans, animals, or plants would come into contact with the composition, are non-toxic and biocompatible.

The term “surfactant” is used to describe composition additives used to solubilize and remove oils and other materials from a surface exposed to the present compositions. Preferred surfactants for use in the present invention are those surfactants which may produce foams (but are not required to) upon exposure to a surface. Some surfactants that may be useful in the present invention include but are not limited to nonionic, anionic, cationic, amphoteric and zwitterionic surfactants. Preferred anionic surfactants for use in the present invention include, for example, alkyl sulfates, alkylether sulfates, alkyl benzene sulfonates, alpha olefin sulfonates, N-alkyl sarcosinates, alkyl sulfosuccinates, alkyl phosphates, alkylether phosphates and alkyl or alkylether carboxylic acid salts, among others. It is understood that surfactants for use in the present compositions that are used with biological membranes or in applications in which humans, animals, or plants would come into contact with the composition, are non-toxic and biocompatible.

The present invention is directed a composition for treatment and control of infection caused by microbes, viruses, fungi, and other pathogens on biological and non-biological membranes and surfaces. The present invention also pertains to a topical composition that has prebiotic and probiotic properties for the control and treatment of microbial infection. Biological membranes support growth of commensal, neutral, as well as pathogenic microbes, and the presently described topical composition has prebiotic and probiotic properties for the control and treatment of infection on biological membranes.

The aqueous composition of the present invention comprises one or more polyol in combination with molecular iodine (I₂) and a source of iodate (IO₃), and an acid to buffer the solution at a pH of between about 1.5 to about 6.5, wherein iodide and iodate are present in the composition at a molar ratio of about 0.1 to about 25, and the one or more polyol is present in the composition in an amount of at least 3% by weight of the composition. The molecular iodine in the composition provides a biocidal effect on pathogenic species, while selection and concentration of the polyol encourage and protect commensal bacteria in oral, nasal, aural, and eye treatment. Preferably the composition comprises one or both of erythritol and xylitol as the polyol. Pathogenic species that can be treated or controlled with the present composition include but are not limited to bacteria, viruses, fungi, mold, and spores. The biocidal utility of the composition is made possible due to the high concentration of molecular iodine constituent of the solution which remains stable when mixed with one or more polyols. The addition of the polyol does not affect the biocidal activity or stability of the molecular iodine in the composition, such as by inactivating it or taking it out of solution, as other additives have been shown to do.

In oral health, a composition comprising molecular iodine in combination with a polyol, preferably erythritol and xylitol, provides antimicrobial biocidal activity with the least effect on the commensal nitrate and nitrite-reducing oral bacteria. The concentration of uncomplexed molecular iodine serves as a disinfectant, biocidal, preservative and/or antimicrobial in the composition, and the polyol provides both pre-biotic and pro-biotic activity to the composition for beneficial bacteria on the membrane being treated. The composition can act as a pre-biotic, pro-biotic, and can be used as both a preventative therapy and a therapeutic treatment to promote growth of beneficial bacteria and act as a biocide for microorganisms including detrimental bacteria, viruses, and fungi. Without being bound by theory, it has been found that the present composition is also known to spare and/or support the commensal and nitrite and nitrate reducing oral bacteria due to the presence of the polyol, and is particularly useful in oral formulations to preserve populations of beneficial oral bacteria. In particular, while simultaneously being selectively biocidal to pathogenic bacteria in humans to advance oral and systemic health, the present composition can preserve and promote a healthy oral microbiome. The activity of the present composition as a result of the stabilized concentration of free molecular iodine in solution in combination with the advantages provided by the polyol provides a prebiotic and probiotic composition with high activity, low environmental impact, long term storage stability, and low toxicity. In addition, the present composition is effective against pathogenic bacteria, while limitedly disrupting essential commensal bacteria, such as nitrate-reducing and nitrite producing bacteria, and in a manner that utilizes naturally occurring ingredients that are already approved for human use.

Compositions of the present invention can be used for treating and/or preventing viral, bacterial (both gram negative and positive), parasite, fungal and spore-based infections, especially including Norovirus, Poliovirus, Coronavirus, Hepatitis A, Klebsiella pneumonie, Staphyloccus aureus, Trichophyton mentagrophytes, Acinetobacter boumanni, and Candida albicans in subjects or patients in need. The present compositions can also be used for treating biological and non-biological surfaces, including keratinous and mucosal tissue surfaces and wounds, represent additional uses of the present invention.

There is a missing link in oral care that is not yet widely appreciated. In particular, eradication of commensal oral bacteria in a broad spectrum, indiscriminate fashion has been found to decrease the nitrate and nitrite reducing bacteria available in the mouth. There are at least fourteen species of oral bacteria that have been found to have substantial nitrate or nitrite-reducing capabilities and these bacteria may provide a mechanism to help to explain the oral systemic link we are searching for. (Nathan S. Bryan et al., (2011) Nitrite and Nitrate in Human Health and Disease pp. 23-31, Humana Press ISBN: 978-1-60761-615-3; E. R. Hyde et al., (2014) Metagenomic Analysis of Nitrate-Reducing Bacteria in the Oral Cavity: Implications for Nitric Oxide Homeostasis. PLoS ONE 9(3): e88645; E. Soderling et al., (2000), Influence of Maternal Xylitol Consumption on Acquisition of Mutans Streptococci by Infants. Journal of Dental Research, 79(3):882-887.) While probiotics can replenish and normalize a microbiome, it is far better not to use products that disrupt commensal oral flora. Polyols act as prebiotics and are protective for these commensal bacteria, while at the same time, they also act to inactivate some of the problematic and pathogenic oral bacteria, including Streptococcus mutans which is indicated as contributing to tooth decay. (Kaumudi J. Joshipura et al., Nitric Oxide, Volume 71, 1 Dec. 2017, Pages 14-20)

In oral compositions, the combination of stabilized molecular iodine and a polyol are effective by inhibiting the specific bacteria that lead to tooth decay, thereby treating the underlying cause of dental caries, as well as supporting the health of commensal oral bacteria. The halogen fluoride, which is traditionally used in oral cane prevention, can be replaced with molecular iodine in combination with polyol to inhibit growth of the specific bacteria that cause tooth decay while preserving the health of commensal oral bacteria. Orally applied polyol has also been demonstrated to have the remarkable ability to remineralize teeth which allows for healing of dental caries or lesions.

The present invention provides compositions of uncomplexed molecular iodine and polyols that are stable, non-irritating, non-toxic and capable of being placed into commercial distribution channels with extended storage stability (months or years). In one embodiment, the compositions described in this application: (a) provide a constant thiosulfate titratable level of iodine over the shelf-life of the product and (b) exhibit a chemical activity of molecular iodine that is at least about 50% (at least about 55%, at least about 60%, at least about 65%, at least about 70%, at least about 75%, at least about 80%, at least about 85%, 90%, 95% or more) of an equivalent concentration of molecular iodine (based upon the total atoms of iodine) as measured potentiometrically in a 0.1 N HCl solution. Thus, the present compositions provide an unexpectedly high level of activity of molecular iodine as an uncomplexed species in combination with a beneficial concentration of polyol in a composition which is storage stable.

Polyols

The amount of polyol in the composition includes a 1%-30% mixture of polyols, especially xylitol and erythritol with the ratio varying on by the purpose of the solution. Preferably the polyol is at least 3% by weight in the concentration, and preferably up to about 30% by weight or more, and more preferably up to 25% by weight of the composition. Polyols are products of photosynthesis and are found in fruits and vegetables, including tubers and leafy green vegetables. More than 10,000 years ago, humans who settled in Sweden chewed gobs of sticky “pitch,” which was a tar like material extracted from Birch bark, a tree that is a good source of xylitol, and the population saw little tooth decay. Polyols can also be produced by certain bacteria, yeasts, algae, and lactic acid bacteria. Polyols include the sugars mannitol, erythritol, sorbitol, dulcitol, inositol, xylitol, lactitol, ribitol, and arabitol. Polyols can affect oral biofilm formation, have dental remineralization potential, and inhibit adherence and growth of bacteria such as oral streptococci. In one study, Soderling et al. showed that 4% of xylitol or erythritol both had effects of inhibiting certain oral pathogenic bacteria and also affected adherence of biofilm. (Eva M. Soderling et al., Current Microbiology 60(1):25-9, September 2009) In a review by Zhan, it was shown that xylitol and erythritol can serve as good components of anti-caries regiments as oral microbiome modifiers, sugar substitutes, and agents to prevent Strep. Mutans transmission. (L. Zhan, Adv Dental Research; 2018, Feb 29, 110-116)

Xylitol has been incorporated into candies, oral and nasal compositions, toothpaste, troches, lozenges, mints, and chewing gum after modern day studies demonstrated that it affected Strep. Mutans and Lactobacillus and lowered caries rates and inhibited oral biofilm formation. Xylitol also tastes great and has a nice cool “mouth feel”. Xylitol also has humectant capabilities that can help with “dry mouth” symptoms, and is also a probiotic and promotes the growth and metabolic activity of butyrate producing bacteria in the large colon. (T. Sato et al., FEMS Microbiol Ecol. 2017 January;93(1)) Nitrate reducing bacteria are also encouraged with xylitol and erythritol, and it has been shown that salivary bacterial nitrates are protective against caries (J. J. Doetl et al., European Journal of Oral Sciences. 2004 Oct;112(5):424-8) On skin, xylitol has been shown to improve barrier function and suppress the growth of certain potential skin pathogens. (K. Salli, et al. Nutrients. 2019 August; 11(8): 1813)

Erythritol is a four-carbon polyol which occurs naturally in small quantities in alcoholic beverages, mushrooms, and fruits such as pears, grapes, and watermelons. Erythritol has been shown to be beneficial when used in oral composition for lowering plaque growth, reducing local concentrations of acetic acid and propionic acid, and decreasing streptococcus Mutans over a three-year study. (P. de Cock et al., International Journal of Dentistry, Volume 2016, Article ID 9868421, 15 pages). In vitro studies indicated that erythritol impedes biofilm formation with Streptococcus mutans, oral inhabitant Streptococcus gordonii, and the periodontal pathogen Porphyromonas gingivalis (D. A. Rzechonek et al., Critical Reviews in Biotechnology, Volume 38, 2018-Issue 4). The combination of xylitol and erythritol has also been shown to have both singular and synergistic effects in the oral cavity. (S. Koljalg et al., Scientific Reports, Vol. 10, Article number: 6297, 2020).

Molecular Iodine

Molecular iodine is an I₂ species in an aqueous environment. Molecular iodine is used in the present composition as a biocidal and antimicrobial. In an aqueous environment iodine exists in several forms or species. These species include iodide (11, molecular iodine (I₂), hypoiodous acid (HOI), iodate (IO₃⁻), triiodide (I₃⁻) and polyiodides (e.g., I₅⁻ or I₇⁻). Each of these species have different physical and chemical properties. While molecular iodine has been historically unstable in aqueous solution and can be inactivated by many additives, Kolsky et al. have described formulations of uncomplexed molecular iodine that are stable, non-irritating, non-toxic and capable of being placed into commercial distribution channels with extended storage stability (months or years). (United States patent U.S. Pat. No. 10,092,006)

In the present composition, iodate generates molecular (uncomplexed) iodine within the composition for use as a disinfectant/biocide/antimicrobial pursuant to the present invention. By relying on iodate to produce uncomplexed molecular iodine on an ongoing basis, the concentration of uncomplexed molecular iodine within the present composition remains within a range of concentration which maximizes biocidal/germicidal and disinfectant activity for unexpectedly long periods of time. For example, depending upon the amount of acid and the buffered pH of the composition (note that a lower pH within the range of the present compositions will tend to result in greater stability and a more efficient formation of molecular iodine from iodate than a higher pH), from at least a week to about 5 years, about two-three weeks to about 3-4 years, about a month to about 2-2.5 years, or at least 3-6 months to a year within this range.

It has been further demonstrated that the use of polyols has little to no effect on the biocidal activity of the iodine or the stability of the iodine in solution. By marrying together stabilized molecular iodine with one or more polyol in the composition of this invention, the beneficial effects of both species can be obtained while at the same time only minimally disturbing the vital and essential commensal oral/nasal bacteria.

Molecular iodine (I₂) and a source of iodate (IO₃) are present in the composition in a molar ratio of about 0.1 to about 25, or preferably from about 1.5 to about 5.0, about 0.25 to about 10, about 1.25 to 5.0, about 1.0 to 7.5, about 1.25 to 5.0, about 1.25 to about 5.0, or more preferably from about 1.5 to about 5.0. The concentration of uncomplexed molecular iodine (I₂) acts as a disinfectant, biocide and/or antimicrobial and is effective in an amount in the composition ranging from about 0.5 ppm to about 2500 ppm, and is preferably in the composition in an amount of between about 1 ppm to about 1000 ppm, about 10 pm to about 500 ppm, about 20 ppm to about 350 ppm and about 25 ppm to about 300 ppm, about 35 ppm to about 250 ppm, about 50 ppm to about 200 ppm, the concentration of acid in the composition is effective to provide a buffering pH in the composition ranging from about 1.5 to about 6.5 (often about 2.0 to about 6.5 within this range), preferably 2.0 to about 5.5, often about 2.0 to about 5.0). An effective concentration of biocompatible acid in the composition, either inorganic or organic, is effective to provide a buffering pH in the composition. One or more polyols are provided in the range of 3-50% of the total solution to enhance taste as well as for their biological properties. Preferable polyols are xylitol and erythritol, individually or in combination, at a concentration of about 1-30% by weight of the composition, more preferably between 3-25% by weight.

Compositions according to the present invention can be stored stably for unexpectedly long periods of time, up to about 2-5 years, and at least for about 2-5 weeks and up to years without refrigeration at ambient temperatures. Iodine concentration measurements of the present compositions including polyol have remained stable over a period of two years with little or very low reduction in iodine concentration in solution. It is noted that extreme heat and direct light can cause degradation of the iodine, however the stability of the iodine is not significantly affected by the addition of the polyol to the composition.

Uses of the Composition

The present composition can be used as a disinfecting solutions in mucous membranes, in biological cavities, and topically on skin, specifically as a germicide and/or biocide (e.g. antiviral, antibacterial, antifungal, antispore etc.). The composition can be used as a disinfectant, sanitizer, sterilants, and sporicides to kill viruses, fungi, bacteria, spores, mold and other known microbes. The present invention may be used in various applications or general uses, among others without limitation as disinfectants, sanitizers, antimicrobial agents and/or biocides.

The present compositions can be used in the mouth, nose, eyes, ears, or other mucous membrane or body cavity, as well as topically on skin, keratinous tissue, or mucosal tissue including subgingival pockets and periodontal spaces, periodontal surface. When used as a dental compositions the formulation can be prepared as pre-procedure or post-procedure rinse, mouthwash, spray, drop, or other formulation. The composition can also be used as an additive on dental floss, for example use on tooth, gum, and tongue as an aid to cleaning the mouth and tongue. The present composition can also be used to coat medical and personal cleaning implements such as, for example, floss, wipes, cotton swabs, Q-tips™, brushes, tongue cleaners, toothpicks, interproximal cleaners, applicators, and other devices for topical and surface disinfection. For application to the nose the composition can be formulated, for example, as a nasal drop, nasal spray, nasal foam, inhalant, troche, or in any other suitable formulation.

In addition to antibacterial and antiviral uses, the compositions according to the present invention may be also be used as antifungal agents to eliminate or disinfect numerous types of disease causing fungi including Aspergillus, Coccidioides, Histoplasma capsulatum, and Candida, including Candida albicans, among others. The present invention may also be used to inhibit and/or eliminate spores and mold. The present invention may be used to substantially inhibit and/or eliminate spores from numerous surfaces in numerous applications as otherwise described herein. The present invention is useful to inhibit and/or eliminate each of these molds from surfaces on or in solutions in which they are present. The present invention may also be used to inhibit and/or eliminate prions, which are a class of bioagents comprised of misfolded proteins with the ability to transmit their misfolded shape onto normal variants of the same protein. Exemplary prions include Scrapie (Sheep and goats), transmissible mink encephalopathy (TME), chronic wasting disease (CWD) in mule deer and elk, bovine spongiform encephalopathy (BSE) cattle, feline spongiform encephalopathy (FSE) in cats, exotic ungulate encephalopathy (EUE), Kuru in humans, Creutzfeldt-Jakob disease (CJD) in humans, Fatal familial insomnia (FFI) in humans and Gerstmann-Straussler-Scheinker syndrome (GSS) in humans.

Parasites are another class of important bioagents which may be treated or disinfected using the invention of the present application. Exemplary parasites which can be disinfected (eliminated) by compositions according to the present invention include the following: Alveolar Echinococcosis (Echinococcosis, Hydatid Disease), Angiostrongyliasis (Angiostrongylus Infection), Anisakiasis (An isakis Infection, Pseudoterranova Infection), Ascariasis (Ascaris Infection, Intestinal Roundworms), Babesiosis (Babesia Infection), Balantidiasis (Balantidium Infection), Balamuthia, Baylisascariasis (Baylisascaris Infection, Raccoon Roundworm), Blastocystis hominis Infection, Cercarial Dermatitis (Swimmer's Itch), Chagas Disease (American Trypanosomiasis), Chilomastix mesnili Infection (Nonpathogenic [Harmless] Intestinal Protozoa), Clonorchiasis (Clonorchis Infection), Cryptosporidiosis (Cryptosporidium Infection), Cyclosporiasis (Cyclospora Infection), Cysticercosis (Neurocysticercosis), Cystoisospora Infection (Cystoisosporiasis) formerly Isospora Infection, Dientamoeba fragilis Infection, Diphyllobothriasis (Diphyllobothrium Infection), Dirofilariasis (Dirofilaria Infection), DPDx, Fasciolopsiasis (Fasciolopsis Infection), Foodborne Diseases, Kala-azar (Leishmaniasis, Leishmania Infection), Keratitis (Acanthamoeba Infection), Microsporidiosis (Microsporidia Infection), Myiasis, Naegleria Infection, Neurocysticercosis (Cysticercosis), Neglected Tropical Diseases, Opisthorchiasis (Opisthorchis Infection), Paragonimiasis (Paragonimus Infection), Pneumocystis jirovecii Pneumonia, Pseudoterranova Infection (Anisakiasis, Anisakis Infection), Sappinia, Scabies, Soil-transmitted Helminths, Strongyloidiasis (Strongyloides Infection), Swimmer's Itch (Cercarial Dermatitis), Taeniasis (Taenia Infection, Tapeworm Infection), Toxoplasmosis (Toxoplasma Infection), Waterborne Diseases, and Zoonotic Diseases (Diseases spread from animals to people).

When used as a mouthwash, the present compositions can be used as a dental preprocedural rinse to reduce populations of deleterious bacteria and reduce post-procedure infection. This type of mouthwash can also be advantageously used by dental patients prior to any procedure (e.g., a treatment procedure including a surgery, dental cleaning and/or prophylaxis) to reduce or knock down the microbial load to help reduce the likelihood (prevent) disease transmission from the patient to the dental staff, for example, when the microbes become aerosolized due to water spray, air spray, ultrasonic instrumentation or the spray from a high speed hand piece utilized in dental procedures. In other embodiments, the present compositions can be used to disinfect areas where bone is denuded, especially in the oral environment as in osteonecrosis and radio osteonecrosis. In this embodiment, the concentration of molecular iodine ranges from about 3 to about 600 ppm, often about 5 to about 350 ppm, more often about 10 to about 100 ppm.

The present compositions are useful in other oral, medical, and dental applications such as, for example, as a pre-procedural rinse (dental office), intra-oral irrigation (for use with oral irrigators such as WaterPie), sub-gingival irrigation or infusion (dental office professional use), biofilm remediation, hand scrub (surgical pre-operative or general disinfectant), antiseptic, pre-operative patient surgical antiseptic, ear drops or ear rinse, eye drops, contact lens solution, throat gargle, throat spray, and oral ingestion such as for gastrointestinal diseases. Other medical formulations containing the present composition can include but are not limited to vaginal douche, sinus spray (or rinse), nasal spray (or rinse), impregnated chewing gum, impregnated mouth melts, impregnated lozenges, toothpaste, inhalation mist, inhalers, vaporizers, urinary bladder lavage, abdominal or thoracic cavity lavage, skin and scalp treatment, athlete's foot soak, eyewash, teat dip, vaginal cream, ophthalmic ointment, and colonic irrigation fluid.

The present compositions can also be used in a wide variety of medical, biological, and hygiene applications where disinfection of biological surfaces or environments is desired, including for use in and for humans, animals, in agriculture, or on surfaces. In particular, the present composition can be used as a treatment for biological membranes to act as a biocidal for deleterious microbes while preserving or encouraging populations of commensal microbes. Some examples of use include but are not limited to disinfecting dental cavities and membranes, root canal sealer and irrigant, egg disinfection, fish roe disinfection, surgical wound closure, iodine releasing soaps, treatment of herpes infections, periodontal rinse, trans-tympanic (ear drum) injections for otitis media, iodine releasing drains, burn spray or other treatment composition, ear drains (tubes), periodontal (subgingival) bioresorbable polymer, blood dialysis, tissue and organ transplants and grafts, iodine impregnated tissues, iodine tablets for systemic viral infections, Iodine releasing anti-inflammatory (steroidal) ointments and creams, underarm deodorants, impregnated dental fillings, shampoos, dental dry socket treatment, pericoronitis, female breast nipple infections, skin graft infections, combined with monoclonal antibodies for viral targeting, cold and flu preventive, dental and other plumbing water lines (biofilm preventive), oral mucositis, foot fungus issues, tongue cleaning solutions, wound dressing.

The present compositions can also be used as a surface treatment on non-biological surfaces of medical devices. Medical devices can also be impregnated, treated, and/or covered with the present compositions. Some non-limiting examples which can be treated include catheters, ports, face masks, sanitary pads, tampons, dental floss, wound dressings and band-aids, rectal wipes, vaginal wipes, diapers, and condoms, among others. The present compositions can also be used to disinfect medical devices and non-biological surfaces such as, for example, dialysis equipment, intravenous equipment, blood draw equipment, needles, dressings, surgical instruments, and surgical fabrics such as linens and clothing.

The present compositions can also be used in cleaning and disinfection of non-animal and industrial and commercial surfaces such as, for example, surfaces and environments in medical laboratories, dental laboratories, surgical suites, hospital rooms, residential buildings, kitchens, food preparation areas, abattoirs, building mold remediation, humidifiers, dehumidifiers, air conditioning systems, washrooms, public use areas, dishwashers, food-related appliances, public use rooms such as classrooms and waiting rooms, other public use rooms and buildings, and other environments and surfaces that benefit from disinfection. Other industrial uses include but are not limited to government and military use (combating bioterrorism), veterinary use, horticulture, tattoo parlors, food handling, food transportation, food preparation, and other uses.

As such, depending on the formulation, the present compositions can be use as low level hard surface disinfectants, intermediate level hard surface disinfectants, hospital grade hard surface disinfectants, as a sporicide for hard surfaces or medical/dental equipment and instruments, high level disinfectant, liquid chemical sterilant, hand sanitizer, hand wash, hand rub, food contact surface sanitizer, and dairy sanitizer. In food and agriculture, the present compositions can also be used for the prevention of food spoilage and extension of shelf-life for fruits, vegetables, meats, dairy, seafood and grains, carcass wash, poultry dip, flower vase life extender, food spoilage retardant, food sanitizer, dish and utensil sanitizer (for manual and automatic cleaning), fruit and vegetable cleaner and sanitizer, meat sanitizer, fish sanitizer, grain sanitizer, vegetable sanitizer, fruit sanitizer, water disinfection, pool disinfection, aquaculture, animal husbandry, agriculture, seafood processing, dairy production, breweries, and meat packing. The present compositions may also be useful in other environmental industries such as, for example, in water distribution and oil field biofilm remediation.

The present compositions can also be used as an iodine supplement for treatment of iodine deficiency, such as in oral ingestion for fibrocystic breast disease. As the present compositions can be prepared in ingestibly safe concentrations of the individual components, special formulations can be prepared for safe internal treatment.

Formulations of the Composition

The present compositions can be formulated for application in a variety of forms such as, for example, solid tablets, washes, foam spray products, sprays, gels, liquids, powders, pastes, lozenges, pastilles, foams, gummies, aerosols, mists, and inhaled gases. Other non-limiting specific examples of formulations in which the present compositions can be used are toothpastes, mouthwashes, pastes, creams, lotions, sanitizer gels, solids, aerosols, or sprays. Compositions according to the present invention may be used and stored in a variety of containers given the substantially non-corrosive nature of these compositions.

In one example, the present compositions can also be formulated as a cream or lotion to be applied to the skin or other surface. In such instances, an emulsion can used to formulate the composition. The term “emulsion”, “oil-in-water emulsion” and “water-in-oil emulsion” are used synonymously throughout the specification to describe certain embodiments of compositions according to the present invention. An “emulsion” according to the present invention is a cream or lotion which is generally formed by the suspension of a very finely divided liquid, in this case water, in another liquid, in this case, an oil, or alternatively, an oil, in water. In the present invention, an emulsion is formed when the water phase is compatibilized in an oil phase, such that the water phase becomes dispersed within the oil phase, generally by inclusion of a surfactant or emulsifier. In certain embodiments according to the present invention, the composition takes the form of a powder, tablet or pill that will be added to an aqueous and then utilized for a specific use application. In yet other embodiments the compositions may be orally ingested when configured as a capsule, tablet, chewable tablet, or powder. In another embodiment the composition may be infused subgingivally as a liquid, gel or other medium as a periodontal treatment. Gel formulations of the present composition can be used in a dental composition to be applied to periodontal surfaces as well as mucous membranes, oral and otherwise, and the tongue surface.

The present composition can further comprise one or more additional biocidal or germicidal agents, also referred to as complementary germicides. Complementary germicides can also be added to broaden the spectrum of activity and/or speed of action of the overall biocidal activity of the composition. The term “additional compatible germicide” or “complementary germicide” is used to describe a compatible germicidal agent which may be further included in compositions according to the present invention to enhance the disinfectant/germicidal/antimicrobial activity of compositions according to the present invention without significantly disrupting the activity of the molecular iodine and polyol in the composition. Additional germicidal agents which may be included in compositions according to the present invention include, for example, numerous per-oxygen compounds such as peracetic acid, perborate, peroxides, including hydrogen peroxide and benzoylperoxide, among others, along with alcohols such as ethanol, isopropanol, propanol and saturated octanoic acid. In certain embodiments, caprylic acid may also be included in compositions according to the present invention. The additional germicides are added to the present compositions to enhance the disinfectant/germicidal properties of the present compositions and may also synergistically enhance the germicidal activity of compositions according to the present invention. In one example, peroxide disinfectant can be selected from the group consisting of peracetic acid, hydrogen peroxide, benzoyl peroxide and mixtures thereof.

The composition can also further comprise one or more additives or components selected from the group consisting of non-aqueous solvents, surfactants, emulsifiers, emollients, oils, humectants, conditioning agents, thickeners or thickening agents, gelling agents, bleaching agents, essential oils, medicaments, fragrances, flavouring agents, preservatives, skin protecting agents, pigments, dyes, coloring agents, salts, buffers, and mixtures thereof. Additional components which can be added to compositions according to the present invention include components selected from non-aqueous solvents (ethanol, isopropanol, n-propanol, etc. which also may be included as a secondary germicide), surfactants, emulsifiers, including secondary emulsifiers, emollients, oils, humectants, oils (polar and non-polar), conditioning agents, lubricants, thickeners/thickening agents (including gelling agents), medicaments, fragrances, preservatives, flavoring agents, skin protecting agents, pigments, dyes, coloring agents, and mixtures thereof in order to provide compositions exhibiting characteristics consistent with the use of the compositions, depending upon the surface to be treated, which surfaces include biological surfaces especially including keratinous or mucosal tissue of an animal, including a human. The addition of surfactant, natural or synthetic, has also been found to increase the biocidal efficiency of the present composition. Optional buffers and salts include any medically or topically acceptable salts and buffers which do not or limitedly disrupt the concentrations of molecular iodine and polyol in solution. Examples of acceptable buffers/salts include but are not limited to saline and bicarbonate salts such as sodium bicarbonate. The composition can also include one or more other solvents such as, for example, glycerine, saline, and water with solubilized salts or minerals that are compatible with the other components of the composition.

### EXAMPLES

Mouthwash Composition

For mouthwash products, it is important that the wash targets pathogenic bacteria in the mouth without destroying the good commensal bacteria, especially the nitrate-reducing bacteria and nitrite producing bacteria. It is well established that certain periodontal bacteria are connected with things such as dementia (P. gingivitis), cardiovascular disease, diabetes, death rates and more. The oral microbiome is pivotal in assuring human homeostasis and health and when the oral or nasal flora balance is affected by common mouthwash or nasal use, there can be a systemic price that is paid.

This mouthwash composition can be used as a mouth rinse and throat gargle to inactivate viral particles, including COVID-19 and other harmful pathogens. The composition also supports the growth of commensal oral nitrate-reducing bacteria and nitrite producing bacteria while reducing the populations of deleterious oral bacteria. Iodine is present at an amount of 0.01% in the composition in the form of potassium iodide and potassium iodate.

TABLE 1
Mouthwash Composition

| | | Amount by |
|---|---|---|
| Component | Function | weight % |
| iodine (potassium iodide | antimicrobial | 0.01 |
| and potassium iodate) | | |
| erythritol | antimicrobial | 0.024 |
| xylitol | antimicrobial | 0.024 |
| sodium bicarbonate | pH adjuster/buffer | 0.070 |
| sodium carbonate | pH adjuster/buffer | 0.070 |
| zinc gluconate | chelating agent | 0.030 |
| citric acid | preservative/antimicrobial | 0.2 |
| water | solvent/vehicle | 99.631 |

In addition to acting as an antimicrobial in the composition, the polyols erythritol and xylitol also contribute a sweet flavour, removing the need for addition of other sweeteners and flavours that may have no other function, or deleterious effect in the composition. Addition of various flavours may be used to flavour the mouth wash, providing that the added flavour does not significant affect the concentrations of active molecular iodine and polyol in solution. Some examples of flavours that may be use include but are not limited to mint, cinnamon, licorice, fruit flavours, bubble gum, and any other flavour acceptable for mouthwash or dental products. The addition of glycerin to the composition can further act as a sweetener, dietary suppressant and preservative. The addition of surfactant, natural or synthetic, has also been found to increase the biocidal efficiency. A recommended regimen for usage for children over 6 years of age, adolescents, and adults is twice daily (preferably morning and night) vigorous rinsing with approximately 30 millilitres (1 ounce) of the composition each time for 30 seconds. Other dosage regimens can be prescribed by doctors, dentists, or other medical professionals, to reduce harmful oral microbiological load.

Throat Spray

Throat spray can be useful for reducing pathogenic microbiological load in the mouth and alimentary canal and preventing infection. The following formulations are provided for cinnamon and mint flavoured throat sprays comprising iodine in combination with erythritol and xylitol in 1 litre of water. The ratio of erythritol:xylitol in these example compositions is 1:1, however the ratio can be different, for example, from 1:100 to 100:1. In addition, the composition can comprise only polyols erythritol, only xylitol, or one or more other polyols, optionally in combination with one or more of erythritol and/or xylitol. An acceptable amount of surfactant can also be added to the composition.

TABLE 2
Throat Spray Compositions

| | | Cinnamon | Mint |
|---|---|---|---|
| | | Throat | Throat |
| | | Spray | Spray |
| Component | Function | grams | |
| potassium iodide | antimicrobial | 0.18 | 0.12 |
| potassium iodate | antimicrobial | 0.135 | 0.09 |
| erythritol | antimicrobial | 0.24 | 0.24 |
| xylitol | antimicrobial | 0.24 | 0.24 |
| sodium carbonate | pH adjuster/buffer | 0.75 | 0.75 |
| zinc gluconate | chelating agent | 0.3 | 0.3 |
| citric acid | preservative/antimicrobial | 2.0 | 2.0 |
| flavour | | 4.5 | 3.0 |
| Dowfax ™ 2A1 | surfactant | 0.3 | 0.3 |

Nose Spray

Nose sprays can be used to disinfect the nose and nasal mucous membranes. The following example composition is for preparation of nasal spray in bulk, with amount in grams of each component provided for formulation of 1 litre of spray. Saline is preferably used as the solvent or vehicle to retain isotonicity with nasal mucous membranes and pH is adjusted using a buffer such as sodium carbonate or sodium bicarbonate, or other pharmacologically acceptable buffer, to maintain an acceptable pH. An acceptable amount of surfactant can also be added to the composition.

TABLE 3
Nose Spray Composition

| Component | Function | grams/L |
|---|---|---|
| potassium iodide | antimicrobial | 0.24 |
| potassium iodate | antimicrobial | 0.18 |
| erythritol | antimicrobial | 0.24 |
| xylitol | antimicrobial | 0.24 |
| non-iodized salt (saline) | pH adjuster/buffer | 9.0 |
| citric acid | preservative/antimicrobial | 2.0 |
| Dowfax ™ 2A1 | surfactant | 0.3 |

Oral Gel or Tongue Wash Composition

Tongue wash and oral gel can be used as a topical treatment to treat local infection, prevent infection in dental pockets such as under the gums and between teeth, and to clean the tongue.

TABLE 4
Oral Gel Composition

| Component | Function | grams/L |
|---|---|---|
| potassium iodide | antimicrobial | 0.27 |
| potassium iodate | antimicrobial | 0.19 |
| erythritol | antimicrobial | 0.25 |
| xylitol | antimicrobial | 0.25 |
| sodium carbonate | pH adjuster/buffer | 1.0 |
| zinc gluconate | chelating agent | 0.3 |
| mint extract | flavour | 2.6 |
| citric acid | preservative/antimicrobial | 2.0 |
| xanthan gum | thickener/stabilizer | 20.0 |
| Dowfax ™ 2A1 | surfactant | 0.3 |

Various fragrances that are compatible with the present composition can be used in cosmetic applications, such as in soaps, shampoos, and hand sanitizers. Common fragrances added to these types of products include but are not limited to citrus scents such as lemon, lime, and grapefruit, and floral scents such as rose, jasmine, hibiscus, and vanilla.

All publications, patents and patent applications mentioned in this specification are indicative of the level of skill of those skilled in the art to which this invention pertains and are herein incorporated by reference. The invention being thus described, it will be obvious that the same may be varied in many ways. Such variations are not to be regarded as a departure from the scope of the invention, and all such modifications as would be obvious to one skilled in the art are intended to be included within the scope of the following claims.

1. An aqueous composition comprising:
at least one polyol selected from one or more of xylitol and erythritol, the at least one polyol in an amount of 1-50% by weight of the composition;
uncomplexed molecular iodine (I₂);
a source of iodide (I⁻) in an effective amount;
a source of iodate (IO₃⁻) in an effective amount; and
a buffering acid,
wherein the buffering acid maintains the composition at a pH of between about 2.0 to about 6.5, and
wherein iodide and iodate are present in the composition at a molar ratio of about 0.1 to about 25 and the composition provides a stable concentration of uncomplexed molecular iodine within the range of 0.5 ppm to 2500 ppm.

2. The composition of claim 1, wherein the at least one polyol is present in the composition in an amount of at least 3% by weight of the composition.

3. (canceled)

4. (canceled)

5. (canceled)

6. The composition of claim 1, wherein the at least one polyol in the composition comprises xylitol and erythritol in a 1:1 ratio.

7. The composition of claim 1, wherein the composition further comprises one or more ingredients selected from the group consisting of flavour, color, glycerine, a whitening agent, a bleaching agent, a surfactant, and a breath freshener.

8. The composition of claim 7, wherein the surfactant is selected from one or more of a nonionic surfactant, an anionic surfactant, a cationic surfactant, an amphoteric surfactant, and a zwitterionic surfactant.

9. The composition of claim 7, wherein the surfactant is at least one of an alkyl sulfate, alkylether sulfate, alkyl benzene sulfonate, alpha olefin sulfonate, N-alkyl sarcosinate, alkyl sulfosuccinate, alkyl phosphate, alkylether phosphate and alkyl carboxylic acid salt, and alkylether carboxylic acid salt.

10. The composition of claim 1, wherein the composition is formulated as a spray, inhalant, liquid, gel, or lozenge.

11. The composition of claim 1, wherein the composition is formulated for nasal application, oral application, inhalation, eye application, or aural application.

12. The composition of claim 1, wherein the at least one polyol is xylitol.

13. The composition of claim 1, wherein the source of iodide is selected from sodium iodide, potassium iodide, lithium iodide, calcium iodide, magnesium iodide, hydroiodic acid and mixtures thereof and the source of iodate is selected from the group consisting of sodium iodate, potassium iodate, lithium iodate, calcium iodate, magnesium iodate, and mixtures thereof.

14. The composition of claim 1, wherein the source of iodate is one or more of sodium iodate and potassium iodate.

15. The composition of claim 1, further comprising hydroiodic acid.

16. The composition of claim 1, wherein the concentration of uncomplexed molecular iodine ranges from 1 ppm to 500 ppm in the composition.

17. A method of topical disinfection comprising applying to a surface a composition comprising:
at least one polyol selected from the group consisting of xylitol and erythritol, the at least one polyol in an amount of 1-50% by weight of the composition;
uncomplexed molecular iodine (I₂);
a source of iodide (I⁻) in an effective amount;
a source of iodate (IO₃⁻) in an effective amount; and
a buffering acid;
wherein the buffering acid maintains the composition at a pH of between about 2.0 to about 6.5, and
wherein iodide and iodate are present in the composition at a molar ratio of about 0.1 to about 25 and the composition provides a stable concentration of uncomplexed molecular iodine within the range of 0.5 ppm to 2500 ppm.

18. The method of claim 17, wherein the composition is applied in an oral cavity, nasal cavity, ear, eye, or on skin.

19. The method of claim 17, wherein the surface is a mucous membrane, human surface, animal surface, agriculture surface, biological membrane, dental surface, skin, medical device, commercial surface, laboratory surface, or industrial surface.

20. The method of claim 17, wherein the at least one polyol is, xylitol.

21. Use of a composition comprising:
applying to a surface a disinfecting amount of a composition comprising:
at least one polyol selected from the group consisting of xylitol and erythritol, the at least one polyol in an amount of 1-50% by weight of the composition;
uncomplexed molecular iodine (I₂);
a source of iodide (I⁻) in an effective amount;
a source of iodate (IO₃⁻) in an effective amount; and
a buffering acid,
wherein the buffering acid maintains the composition at a pH of between about 2.0 to about 6.5, and
wherein iodide and iodate are present in the composition at a molar ratio of about 0.1 to about 25 and the composition provides a stable concentration of molecular iodine within the range of 0.5 ppm to 2500 ppm.

22. The use of claim 21, wherein the surface is floss, a wipe, a cotton swab, a Q-tipTM, a brush, a toothbrush, a tongue cleaner, a toothpick, an interproximal cleaner, an applicator, a bandage, tape, or gauze.

23. The use of claim 21 or 22, wherein the use is agricultural, medical, dental, environmental, or veterinary.

24. (canceled)
